(19) United States
(12) Patent Application Publication
(10) Pub. No.: US 2026/0337176 A1
(43) Pub. Date: Oct. 29, 2026

(54) AUTOPHAGY REGULATOR AND USE THEREOF

(71) Applicant: SHENZHEN INSTITUTES OF ADVANCED TECHNOLOGY, CHINESE ACADEMY OF SCIENCES, SHENZHEN (CN)

(72) Inventors: Guofen SONG, SHENZHEN (CN); Huaiyu WANG, SHENZHEN (CN); Penghui LI, SHENZHEN (CN)

(73) Assignee: SHENZHEN INSTITUTES OF ADVANCED TECHNOLOGY, CHINESE ACADEMY OF SCIENCES, SHENZHEN (CN)

(21) Appl. No.: 19/692,605

(22) Filed: May 29, 2026

Related U.S. Application Data
(63) Continuation of application No. PCT/CN2023/135725, filed on Nov. 30, 2023.

(30) Foreign Application Priority Data
Nov. 30, 2023 (WO) PCT/CN2023/135725

(51) Int. Cl. C07D 401/06; A61K 31/4709

(52) U.S. Cl. CPC C07D 401/06; A61K 31/4709

(57) ABSTRACT

The present disclosure discloses an autophagy regulator and use thereof, aiming to solve the problems that autophagy regulators in the prior art exhibit only single targeting and no cell selectivity, resulting in poor efficacy of killing cancer cells. The structural formula of the autophagy regulator is:

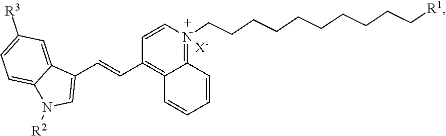

and the autophagy regulator can simultaneously target mitochondria and lysosomes; induce mitophagy, disrupt lysosomes and block autophagic flux; can be used for mitochondrial and lysosomal imaging, and can also be used as an anticancer drug.

### CROSS-REFERENCE TO RELATED APPLICATIONS

The application claims priority to PCT patent application No. PCT/CN2023/135725, filed on Nov. 30, 2023, the entire contents of which are incorporated herein by reference.

### TECHNICAL FIELD

The present disclosure relates to the technical field of organic chemistry and medicine, specifically focusing on an autophagy regulator, along with a preparation method therefor and use thereof.

### BACKGROUND

Autophagy is the self-digestion of lysosomes or vacuoles, and the degradation and recycling of cellular contents. It is critical for maintaining cellular homeostasis and energy balance. Meanwhile, autophagy plays extensive biological roles, including organelle remodeling, protein and organelle quality control, tumor suppression, pathogen elimination, immune and inflammatory regulation, cell survival, and the like. Studies have shown that dysfunction in the autophagy process is associated with various diseases, including cancer, neurodegenerative disease, diabetes, autoimmune disease, cardiovascular disease, and the like. Therefore, autophagy regulation is of great significance for treating various diseases. Currently, autophagy-targeting drugs for various diseases are under further research and development.

Existing autophagy regulators include rapamycin, chloroquine and the like, which can only singly activate autophagy or inhibit autophagy, do not emit fluorescence after binding to targets and cannot be used for fluorescence observation. Particularly when used for tumor treatment, their efficacy and tumor selectivity still require improvement. In addition, an existing related tumor therapeutic drug (Mitochondria/RNA cascade-targeted and fluorescence-switchable photosensitizer for photodynamic therapy augmentation and real-time efficacy self-monitoring, Sensors and Actuators B: Chemical, 369: 132260, 2022 Oct. 15) can only target mitochondria and needs light irradiation to damage mitochondria by producing ROS to induce apoptosis, and then uses light irradiation to induce escape from mitochondria and migration to RNA, after which fluorescence enhances greatly indicating an apoptotic state; in the absence of light irradiation, the drug is only localized in mitochondria and exhibits no obvious cytotoxicity; its anti-tumor application is limited to scenarios involving light irradiation.

### SUMMARY

The present disclosure aims to solve at least one of the technical problems existing in the prior art. To this end, the present disclosure provides an autophagy regulator and use thereof, wherein the autophagy regulator is a dual-targeting fluorescent organic small-molecule autophagy activator and inhibitor, aiming to solve the problems in the prior art that autophagy regulators exhibit single targeting and no cell selectivity resulting in poor efficacy of killing cancer cells, and their use for killing cancer cells relies on light irradiation.

An autophagy regulator according to an embodiment of the first aspect of the present disclosure, the autophagy regulator is a compound having a structure represented by formula (I):

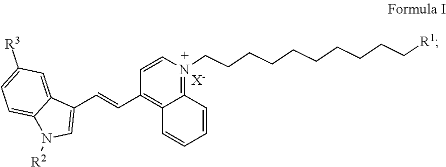

wherein the R¹ is selected from any one of hydrogen or C1-C4 alkyl; the R² is selected from any one of hydrogen or C1-C4 alkyl; the R³ is selected from any one of hydrogen, C1-C4 alkyl, and C1-C4 alkoxy; and the X is selected from any one of halogen atom, BF₄, and ClO₄.

The autophagy regulator is a dual-targeting autophagy activator and inhibitor.

The method for autophagy regulation using the autophagy regulator of the present disclosure is as follows: incubating sample cells with the autophagy regulator in culture medium, wherein the autophagy regulator simultaneously targets mitochondria and lysosomes of the sample cells, damages the mitochondria of the sample cells and induces mitophagy, damages the lysosomes of the sample cells and blocks autophagic flux.

In the step of damaging the mitochondria of the above sample cells by the autophagy regulator in the present disclosure, the mitochondrial membrane potential decreases.

In the step of damaging the lysosomes of the above sample cells by the autophagy regulator in the present disclosure, the lysosomal pH increases.

The autophagy regulator in the present disclosure exhibits high affinity for albumin, and can selectively enter cancer cells after binding to albumin.

The autophagy regulator provided by the present disclosure is a dual-targeting fluorescent organic small-molecule autophagy activator and inhibitor; compared with existing autophagy regulators, the uniqueness of the dual-targeting fluorescent organic small molecule of the present disclosure lies in the fact that it can simultaneously target mitochondria and lysosomes, activate mitophagy, disrupt lysosomal function and block autophagic flux. The autophagy regulator provided by the present disclosure is also a novel mitochondrial/lysosomal fluorescent probe; Compared with existing mitochondrial and lysosomal fluorescent probes, it can simultaneously target mitochondria and lysosomes and emit red fluorescence, image the morphology, number and distribution of mitochondria and lysosomes; Meanwhile it has good membrane permeability and good counterstaining compatibility. The autophagy regulator is also a drug for tumor treatment; Compared with existing drugs for treating tumors, the autophagy regulator exhibits high affinity for albumin, and after binding to albumin, selectively enters cancer cells by active transport, thereby selectively killing cancer cells, and meanwhile, the process of killing cancer cells can be carried out without light assistance.

The dual-targeting fluorescent organic small-molecule autophagy activator and inhibitor provided by the present disclosure, in a first aspect, can serve as an autophagy regulator to target mitochondria and lysosomes, activate mitophagy, and block autophagic flux; in a second aspect, it can serve as a fluorescent probe for labelling mitochondria and lysosomes in cells to visualize their morphology, quantity, and distribution; it can provide a simple, intuitive biological detection reagent for physiological and pathological studies and clinical diagnosis related to mitochondria and lysosomes, and has wide application and good effect. In a third aspect, the autophagy regulator can serve as an anticancer drug, exhibits high affinity for albumin, and after binding to albumin, can selectively enter cancer cells by active transport and kill cancer cells light-independently.

According to some embodiments of the present disclosure, the halogen atom is selected from any one of iodine, bromine and chlorine.

According to some embodiments of the present disclosure, in the R¹, the C1-C4 alkyl includes any one of methyl, ethyl, n-propyl, isopropyl, n-butyl, isobutyl, sec-butyl and tert-butyl.

According to some embodiments of the present disclosure, in the R², the C1-C4 alkyl includes any one of methyl, ethyl, n-propyl, isopropyl, n-butyl, isobutyl, sec-butyl and tert-butyl.

According to some embodiments of the present disclosure, in the R³, the C1-C4 alkyl includes any one of methyl, ethyl, n-propyl, isopropyl, n-butyl, isobutyl, sec-butyl and tert-butyl.

According to some embodiments of the present disclosure, the C1-C4 alkoxy includes any one of methoxy, ethoxy, propoxy and butoxy.

According to some embodiments of the present disclosure, the R¹ is hydrogen, methyl, ethyl, or n-propyl; the R² is selected from hydrogen, methyl, ethyl, and n-propyl; the R³ is selected from hydrogen, methyl, ethyl, n-propyl, methoxy, ethoxy, and n-propoxy.

According to some embodiments of the present disclosure, when R¹ is ethyl, R² is hydrogen, the R³ is methoxy, and the X is iodine, the obtained autophagy regulator is (E)-4-(2-(5-methoxy-1H-indol-3-yl)vinyl)-1-n-dodecylquinolinium iodide.

According to some embodiments of the present disclosure, when R¹ is ethyl, R² is hydrogen, the R³ is hydrogen, and the X is iodine, the obtained autophagy regulator is (E)-4-(2-(1H-indol-3-yl)vinyl)-1-n-dodecylquinolinium iodide.

A preparation method for the autophagy regulator according to an embodiment of the second aspect of the present disclosure, the preparation method comprises the following steps:
S1. dissolving 4-methylquinoline (formula II) and a halogenated long-chain alkane (formula III) in a solvent and refluxing the mixture to generate a 1-long-chain alkyl-4-methylquinolinium salt (formula IV);
S2. dissolving the 1-long-chain alkyl-4-methylquinolinium salt, indole-3-carboxaldehyde (formula V) and a catalyst in a solvent, refluxing the mixture, and then purifying the product;
the catalyst includes piperidine.

According to some embodiments of the present disclosure, the solvent includes ethanol.

According to some embodiments of the present disclosure, the molar ratio of the indole-3-carboxaldehyde to the 4-methylquinoline is 1:(1.0 to 2.0).

According to some embodiments of the present disclosure, in step S1, the time of the reflux reaction is 3 to 4 days.

According to some embodiments of the present disclosure, in step S2, the time of the reflux reaction is 1 to 2 days.

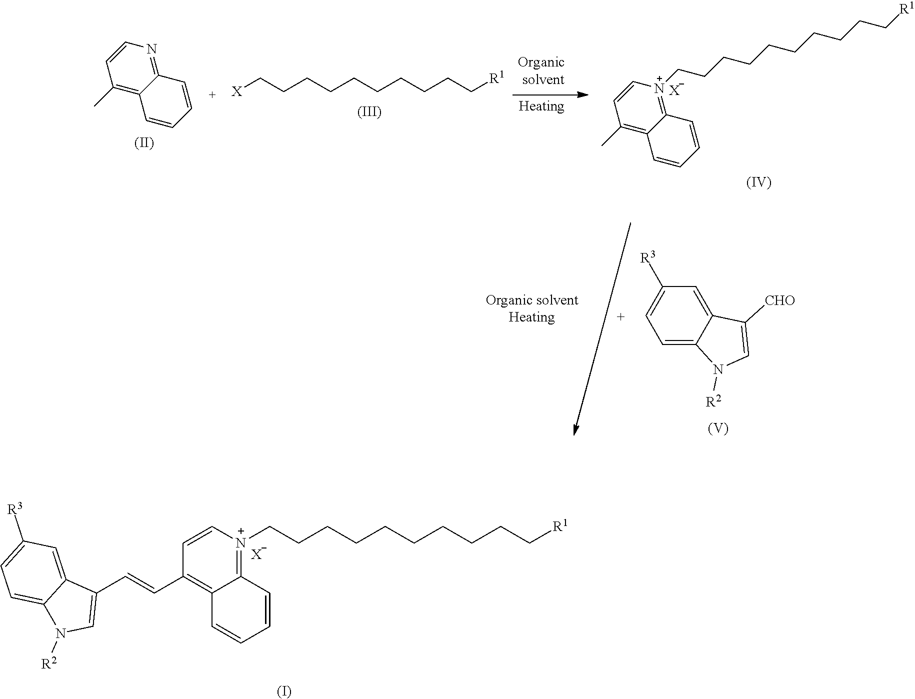

According to some preferred embodiments of the present disclosure, the indole-3-carboxaldehyde is selected from 5-methoxy-3-formylindole and 3-formylindole.

According to some preferred embodiments of the present disclosure, the haloalkane is iodododecane, and using 5-methoxy-3-formylindole and iodododecane as reactants, the preparation method of the autophagy regulator is as follows:
S01. preparing a mixed solution of 4-methylquinoline and iodododecane in ethanol;
S02. refluxing the mixture with stirring for three days;
S03. adding an solution of 5-methoxy-3-formylindole in ethanol;
S04. adding the catalyst piperidine to the mixed solution, refluxing with stirring for one day, and slowly cooling to room temperature to obtain dark green crystals or a crude solid product;
S05. purifying the crude solid product by column chromatography, using dichloromethane/methanol as an eluent, and drying to obtain dark green crystals or dark red powder, wherein the dark green crystals and dark red powder are a dual-targeting fluorescent organic small-molecule autophagy regulator, and the dual-targeting fluorescent organic small-molecule autophagy regulator is (E)-4-(2-(5-methoxy-1H-indol-3-yl)vinyl)-1-n-dodecylquinolinium iodide.

A product for cellular autophagy regulation according to an embodiment of the third aspect of the present disclosure, the product includes the autophagy regulator described above.

A method for applying an autophagy regulator in cellular autophagy regulation according to an embodiment of the fourth aspect of the present disclosure, the method comprises: incubating sample cells with the autophagy regulator, wherein the autophagy regulator simultaneously targets mitochondria and lysosomes in the sample cells, damages the mitochondria of the sample cells and activates mitophagy, damages the lysosomes of the sample cells and blocks autophagic flux.

In the present disclosure, the dual-targeting fluorescent organic small-molecule autophagy activator and inhibitor is used for cellular autophagy regulation excluding diagnostic and therapeutic methods; the dual-targeting fluorescent organic small molecule of the present application can simultaneously target mitochondria and lysosomes of cells, causing the mitochondrial membrane potential to decrease or even dissipate, inducing mitophagy, and generating a large amount of autophagic vesicles; meanwhile, the lysosomal pH rises and lysosomes cannot fuse with autophagosomes to form autolysosomes, so that the completion of autophagic flux is blocked, and dual regulation of autophagy is achieved.

A product for targeting mitochondria and/or lysosomes according to an embodiment of the fifth aspect of the present disclosure, the product includes the autophagy regulator described above.

A product for mitochondrial imaging and/or lysosomal imaging according to an embodiment of the sixth aspect of the present disclosure, the product includes the autophagy regulator described above.

A method for applying the autophagy regulator in mitochondrial imaging and/or lysosomal imaging according to an embodiment of the seventh aspect of the present disclosure, the method comprises: incubating sample cells with the autophagy regulator, wherein the autophagy regulator is localized to mitochondria and/or lysosomes of the sample cells, the fluorescence enhances, and fluorescence imaging of the mitochondria and/or lysosomes is achieved.

The use of a dual-targeting fluorescent organic small-molecule autophagy regulator provided by the present disclosure in mitochondrial/lysosomal fluorescence imaging, compared with existing mitochondrial and lysosomal fluorescent probes, enables the dual-targeting fluorescent organic small molecule to simultaneously target cellular mitochondria and lysosomes; after the molecule is localized to mitochondria and lysosomes, the fluorescence enhances greatly, thereby achieving simultaneous fluorescence imaging of mitochondria and lysosomes.

In the present disclosure, the dual-targeting fluorescent organic small-molecule autophagy regulator is used for mitochondrial/lysosomal fluorescence imaging excluding diagnostic and therapeutic methods; the dual-targeting fluorescent organic small molecule of the present application itself does not emit fluorescence, and after the compound is localized to mitochondria and lysosomes, the fluorescence is greatly enhanced, thereby achieving simultaneous fluorescence imaging of mitochondria and lysosomes.

A drug for tumor treatment according to an embodiment of the eighth aspect of the present disclosure, the drug includes a autophagy-modulating drug, wherein the autophagy-modulating drug includes the autophagy regulator.

According to some embodiments of the present disclosure, the tumor includes a tumor overexpressing an albumin receptor and/or an albumin-binding protein.

According to some embodiments of the present disclosure, the tumor overexpressing the albumin receptor and the albumin-binding protein includes cervical cancer, breast cancer, ovarian cancer, melanoma, pancreatic cancer, liver cancer, and the like.

According to some embodiments of the present disclosure, the drug includes an injectable composition or an oral composition.

According to some embodiments of the present disclosure, the composition includes a dual-targeting fluorescent organic small-molecule autophagy regulator and other pharmaceutically acceptable carriers, wherein the carriers include, but are not limited to, various pharmaceutical excipients.

A method for killing cancer cells according to an embodiment of the ninth aspect of the present disclosure, the method comprises: incubating sample cells with the autophagy regulator in an albumin-containing medium, wherein the autophagy regulator binds to albumin due to high affinity for albumin, and selectively enters cancer cells in the sample cells by active transport, simultaneously targets mitochondria and lysosomes in the cancer cells, activates mitophagy, and blocks lysosomal function and autophagic flux, inducing death of the cancer cells.

Compared with existing autophagy regulators, the autophagy regulator in the present disclosure, namely the dual-targeting fluorescent organic small-molecule autophagy activator and inhibitor, has a dual-targeting effect of simultaneously targeting cellular mitochondria and lysosomes, activates mitophagy and disrupts lysosomal function, ultimately causing cell death. The dual-targeting fluorescent organic small-molecule autophagy activator and inhibitor can be used to kill cancer cells, thereby achieving use of the dual-targeting fluorescent organic small-molecule autophagy activator and inhibitor in autophagy regulation and cancer cell killing.

### BRIEF DESCRIPTION OF DRAWINGS

The above and/or additional aspects and advantages of the present disclosure will become apparent and readily understood from the following description of examples with reference to the accompanying drawings, wherein:

FIG. 1 shows fluorescence micrographs of HeLa cells co-stained with (E)-4-(2-(5-methoxy-1H-indol-3-yl)vinyl)-1-n-dodecylquinolinium iodide, a mitochondrial green fluorescent probe (MitoTracker Green) and a lysosomal deep-red fluorescent probe (LysoBrite NIR) provided in Test example 2 of the present application.

FIG. 2 shows fluorescence micrographs of HeLa cells stained with rhodamine 123 after treatment with (E)-4-(2-(5-methoxy-1H-indol-3-yl)vinyl)-1-n-dodecylquinolinium iodide provided in Test example 3 of the present application.

FIG. 3 shows fluorescence micrographs of HeLa cells stained with a lysosomal green fluorescent probe (Lysosensor Green DND-189) after treatment with (E)-4-(2-(5-methoxy-1H-indol-3-yl)vinyl)-1-n-dodecylquinolinium iodide provided in Test example 4 of the present application.

FIG. 4 shows fluorescence micrographs of HeLa cells stained with 5-chloromethylfluorescein diacetate (green live-cell tracking probe CellTracker™ Green CMFDA) after treatment with (E)-4-(2-(5-methoxy-1H-indol-3-yl)vinyl)-1-n-dodecylquinolinium iodide provided in Test example 5 of the present application.

FIG. 5 shows staining fluorescence micrographs of mitochondria and lysosomes in HeLa cells after individual treatment with (E)-4-(2-(5-methoxy-1H-indol-3-yl)vinyl)-1-n-dodecylquinolinium iodide and CCCP provided in Test example 6 of the present application.

FIG. 6 shows fluorescence micrographs of HeLa cells after incubation with (E)-4-(2-(5-methoxy-1H-indol-3-yl)vinyl)-1-n-dodecylquinolinium iodide at 37 °C and 4 °C, respectively, provided in Test example 7 of the present application.

FIG. 7 shows micrographs of normal cell spheroids HEK293 and cancer cell spheroids HeLa before and after treatment with (E)-4-(2-(5-methoxy-1H-indol-3-yl)vinyl)-1-n-dodecylquinolinium iodide provided in Test example 8 of the present application.

FIG. 8 shows fluorescence spectra and double-logarithmic fitting curves of interaction between (E)-4-(2-(5-methoxy-1H-indol-3-yl)vinyl)-1-n-dodecylquinolinium iodide and albumin provided in Test example 9 of the present application.

FIG. 9 shows fluorescence micrographs of cancer cells and normal cells after incubation with (E)-4-(2-(5-methoxy-1H-indol-3-yl)vinyl)-1-n-dodecylquinolinium iodide and (E)-4-(2-(5-methoxy-1H-indol-3-yl)vinyl)-1-n-octylquinolinium iodide provided in Test example 10 of the present application.

FIG. 10 shows fluorescence micrographs of live HeLa cells after incubation with (E)-4-(2-(5-methoxy-1H-indol-3-yl)vinyl)-1-n-octylquinolinium iodide at 37 °C and 4 °C, respectively, provided in Test example 11 of the present application.

FIG. 11 shows co-localization fluorescence images of (E)-4-(2-(1H-indol-3-yl)vinyl)-1-n-dodecylquinolinium iodide with a commercial mitochondrial green fluorescent probe and a lysosomal near-infrared fluorescent probe in HeLa cells provided in Test example 12 of the present application.

FIG. 12 shows fluorescence images of CellTracke Green in HeLa cells after treatment with (E)-4-(2-(1H-indol-3-yl)vinyl)-1-n-dodecylquinolinium iodide provided in Test example 13 of the present application.

### DETAILED DESCRIPTION OF THE EMBODIMENTS

To clarify the technical problems addressed, technical solutions proposed and the beneficial effects of the present application, a detailed description is provided below with reference to specific examples. It should be noted that the examples presented herein are intended solely for illustrative purposes and should not be construed as limitations on the scope of the present application.

In the present application, the term “and/or” describes the associative relationship between related objects, indicating the potential existence of three distinct relationships. For example, A and/or B may refer to: the situation where A exists independently, the situation where both A and B coexist, or the situation where B exists independently. Both A and B can be singular or plural. The character “/” typically represents an “or” relationship between the associated objects.

In the present application, “at least one” refers to one or more, and “a plurality of” means two or more. “at least one of the following” or similar expressions refer to any combination of these items, including both single and plural selections. For example, expressions like “at least one of a, b, or c”, or “at least one of a, b, and c” can signify the following combinations: a, b, c, a-b (i.e., a and b), a-c, b-c, or a-b-c, wherein a, b and c may each be single or multiple.

It should be noted that in various embodiments of the present application, the sequence numbers of the above processes do not imply a specific execution order. Some or all steps may be executed in parallel or sequentially. The execution order of each process should be determined based on its function and internal logic, and should not impose any limitations on the implementation processes of the embodiments of the present application.

The terminology used in the embodiments of the present application is for describing particular embodiments only, and is not intended to limit the scope of the present application. The singular forms “a”, “an”, “the” and “said” used in the embodiments and the appended claims include plural referents, unless the context clearly dictates otherwise.

The weight of relevant components mentioned in the specification of the embodiments of the present application may refer not only to the specific content of each component, but also to the weight ratio of the components. Therefore, any proportional increase or decrease of said amounts falls within the scope disclosed herein. Specifically, the mass values may be expressed in μg, mg, g, kg or other mass units conventional in chemistry.

### Example 1

This example provides the synthesis of (E)-4-(2-(5-methoxy-1H-indol-3-yl)vinyl)-1-n-dodecylquinolinium iodide:

First, 200 μL of 4-methylquinoline and 393 μL of iodododecane were dissolved in 10 mL of ethanol, and the mixture was stirred and refluxed at 85 °C for three days. Then, an ethanol solution containing 0.263 g of 5-methoxy-3-formylindole and five drops of piperidine was added sequencely. The mixture gradually turned red and refluxed for one day. At last, the mixture was slowly cooled, and the precipitate was filtered and washed with a small amount of isopropanol as dark green crystalline grains and dark red powder. Or excess solvent in mixture was evaporated and the crude product was purified by column chromatography using dichloromethane/methanol as an eluent. The dark green crystals and dark red powder were obtained with a yield of ~27%.

¹H NMR (400 MHz, DMSO-d₆), δ (ppm): 12.11 (s, 1H), 9.12 (d, J=4.0 Hz, 1H), 8.97 (d, J=8.0 Hz, 1H), 8.63 (d, J=16.0 Hz, 1H), 8.44 (m, 3H), 8.18 (t, J=8.0 Hz, 1H), 7.99 (m, 2H), 7.70 (s, 1H), 7.42 (d, J=8.0 Hz, 1H), 6.90 (dd, J=4.0, 8.0 Hz, 1H), 4.85 (t, J=8.0 Hz, 2H), 3.90 (s, 3H), 1.91 (m, 2H), 1.37 (m, 2H), 1.21 (m, 16H), 0.83 (t, J=8.0 Hz, 3H). ¹³C NMR (400 MHz, DMSO-d₆), δ (ppm): 155.71, 154.17, 146.15, 139.13, 138.31, 135.12, 132.60, 132.44, 128.80, 127.28, 126.96, 126.15, 119.29, 115.08, 113.87, 113.81, 112.95, 112.80, 102.69, 56.18, 56.07, 31.76, 29.66, 29.47, 29.36, 29.33, 29.18, 28.98, 26.27, 22.57, 14.43. FIRMS: calculated 469.32, found 469.32.

### Example 2

This example provides the synthesis of (E)-4-(2-(1H-indol-3-yl)vinyl)-1-n-dodecylquinolinium iodide (i.e., R¹ is ethyl, R² is hydrogen, and R³ is hydrogen):

200 μL of 4-methylquinoline and 370 μL of iodododecane were dissolved in 10 mL of ethanol, and the mixture was stirred and refluxed at 85 °C for three days. Then, an ethanol solution containing 0.225 g of 3-formylindole was added, followed by addition of several drops of piperidine, The solution gradually turned red and was refluxed for one day. Last, excess solvent was evaporated, and the crude product was purified by column chromatography using dichloromethane/methanol as an eluent. Reddish-brown powder was obtained with a yield of ~21%.

¹H NMR (400 MHz, DMSO-d₆), δ (ppm): 12.13 (s, 1H), 9.10 (d, J=8.0 Hz, 1H), 8.93 (d, J=8.0 Hz, 1H), 8.55 (d, J=16.0 Hz, 1H), 8.39 (m, 3H), 8.22 (t, J=4.0 Hz, 1H), 8.15 (t, J=8.0 Hz, 1H), 7.96 (m, 2H), 7.50 (m, 1H), 7.25 (m, 2H), 4.82 (t, J=8.0 Hz, 2H), 1.88 (m, 2H), 1.35 (m, 2H), 1.17 (m, 16H), 0.79 (t, J=8.0 Hz, 3H). ¹³C NMR (400 MHz, DMSO-d₆), δ (ppm): 154.12, 146.19, 139.32, 138.17, 137.83, 135.14, 133.57, 128.93, 126.92, 126.10, 125.81, 123.56, 122.00, 120.80, 119.18, 115.12, 113.93, 113.08, 113.05, 56.27, 31.72, 29.63, 29.43 (2C), 29.33, 29.13, 28.95, 26.23, 22.52, 14.36. FIRMS: calculated 439.31, found 439.31.

In order to expand more compounds with similar functions in the present disclosure, R³ may also be selected from any one of hydrogen, C1-C4 alkyl, and C1-C4 alkoxy, and R² is selected from any one of hydrogen or C1-C4 alkyl while keeping the molecular parent core skeleton unchanged. According to studies by the inventors, the function of the autophagy regulator in the present disclosure is determined by a conjugated organic cation group and is unrelated to an anion X⁻; R² is selected from any one of hydrogen or C1-C4 alkyl; R³ is selected from any one of hydrogen, C1-C4 alkyl, and C1-C4 alkoxy, and changes within this range do not affect the function of the molecule. However, the length of the carbon chain connected to R¹ has a relatively great influence on the targeting property and protein affinity of the molecule; for example, indole octyl quinolinium salt has weak affinity for albumin and still enters cells via free diffusion, and as the quinoline carbon chain increases (from decyl to tetradecyl), the affinity for albumin of compounds is enhanced and they enter cells by the way of active transport.

### Test Example 1

### Culture of HeLa, 4T1 and HEK293 Cells and Cell Spheroids:

Cancer cells HeLa and 4T1 and normal cells HEK293 were cultured using a complete medium (DMEM medium containing 10% fetal bovine serum and 1% penicillin/streptomycin) in a saturated-humidity incubator at 37 °C and 5% CO₂, and passaged once every 2 to 3 days.

When the cells grew to the logarithmic phase, they were transferred to confocal dishes or 96-well plates: Cells fully grown in a T25 cell culture flask were first washed with PBS, and then digested with 1 mL of trypsin for 1 to 2 minutes (0.25% trypsin for HeLa and 4T1, and 0.025% trypsin for HEK293). After removal of trypsin, fresh medium was added, and the cells were pipetted uniformly and counted, whose density was controlled by the amount of medium added. (1) The cells were inoculated into a confocal glass-bottom culture dish, and was cultured in a 5% CO₂ incubator.When the cells grew to a coverage of ~70% they were used for confocal imaging experiments. (2) The cells were inoculated into a low-attachment U-bottom 96-well plate, and when the diameter of the cell spheroids reached ~800 μm, the cell spheroids were used for antitumor evaluation.

### Test Example 2

### Localization Observation of (E)-4-(2-(5-Methoxy-1H-Indol-3-Yl)Vinyl)-1-n-Dodecylquinolinium Iodide in HeLa Cells

HeLa cells grown on confocal glass-bottom culture dishes prepared in Test example 1 was washed twice with PBS, and then the following staining steps were carried out: (1) incubation with a 0.5 μM commercial mitochondrial green fluorescent probe (MitoTracker Green) solution for 30 min, followed by PBS washing; (2) incubation with a 0.5 μM commercial lysosomal near-infrared fluorescent probe (LysoBrite NIR) solution for 30 min, followed by PBS washing; (3) incubation with a 2 μM (E)-4-(2-(5-methoxy-1H-indol-3-yl)vinyl)-1-n-dodecylquinolinium iodide solution for 30 min, followed by DMEM washing. The stained cell samples were subjected to multichannel fluorescence colocalization observation using a confocal fluorescence microscope.

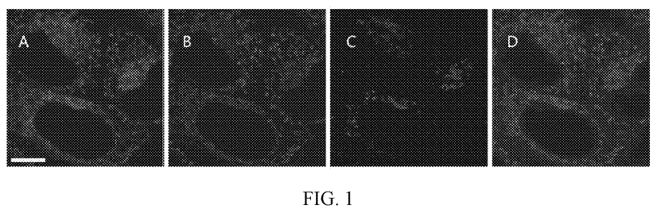

The results are shown in FIG. 1, wherein FIG. 1(A) shows a red fluorescence image of the compound synthesized in Example 1, FIG. 1(B) shows a fluorescence image of the commercial MitoTracker Green, FIG. 1(C) shows a fluorescence image of the commercial LysoBrite NIR, and FIG. 1(D) shows an overlay image of FIG. 1(A), FIG. 1(B) and FIG. 1(C). FIG. 1(A) covers both regions of FIG. 1(B) and FIG. 1(C), and exhibits excellent overlap with the combined signal of FIG. 1(B) and FIG. 1(C), indicating that the fluorescence of the compound synthesized in Example 1 is distributed in the two organelles, mitochondria and lysosomes. This result demonstrates that (E)-4-(2-(5-methoxy-1H-indol-3-yl)vinyl)-1-n-dodecylquinolinium iodide can be used for targeting mitochondria and lysosomes as well as simultaneous fluorescence imaging of mitochondria and lysosomes.

### Test Example 3

### Changes in Mitochondrial Membrane Potential of HeLa Cells Before and After Treatment With (E)-4-(2-(5-Methoxy-1H-Indol-3-Yl)Vinyl)-1-n-Dodecylquinolinium Iodide

Two groups of HeLa cells grown on confocal glass-bottom culture dishes prepared in Test example 1 were both incubated with 5 μM rhodamine 123 in darkness in a CO₂ incubator for 30 min and then washed with PBS. After that, one group was incubated in the dark for another 30 min in complete medium with 10 μM of the compound synthesized in Example 1, and the other group was incubated in complete medium with an equal amount of DMSO as a blank control sample. After PBS washing, the cells were observed under a laser scanning confocal microscope, and the changes of the fluorescence brightness from rhodamine 123 in the two groups of cells were recorded.

### Result Analysis:

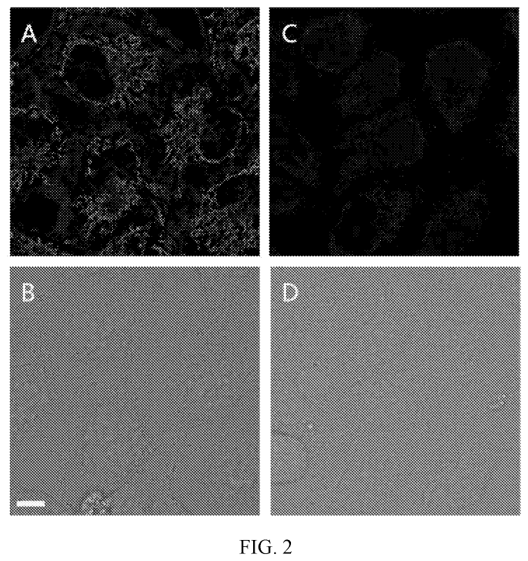

The experimental results of Test example 3 are shown in FIG. 2. FIG. 2 shows fluorescence micrographs of rhodamine 123, a mitochondrial membrane potential probe, in HeLa cells before and after treatment with the molecule synthesized in Example 1. FIG. 2(A) shows a fluorescence micrograph of rhodamine 123 in cells of the blank control sample group; FIG. 2(B) shows a bright-field micrograph corresponding to FIG. 2(A); FIG. 2(C) shows a fluorescence micrograph of rhodamine 123 in the cells treated with the molecule synthesized in Example 1; FIG. 2(D) shows a bright-field micrograph corresponding to FIG. 2(C). It can be learned from FIG. 2(A) and FIG. 2(C) that, in the cells treated with the compound synthesized in Example 1, the fluorescence of rhodamine 123 is greatly weakened (FIG. 2(C)). This represents that the mitochondrial membrane potential of the cells treated with the compound synthesized in Example 1 decreases significantly, indicating mitochondrial damage.

### Test Example 4

Changes in Lysosomal pH of HeLa Cells Before and After Treatment with (E)-4-(2-(5-Methoxy-1H-Indol-3-Yl)Vinyl)-1-n-Dodecylquinolinium Iodide

Two groups of HeLa cells grown on confocal glass-bottom culture dishes prepared in Test example 1 were incubated with 2 μM lysosomal green fluorescent probe (LysoSensor Green DND-189) in the dark for 30 min in a CO₂ incubator, and then washed with PBS. After that one group was incubated in the dark for 30 min in complete medium with 10 μM of the compound synthesized in Example 1, and the other group was incubated in complete medium with an equal amount of DMSO as a blank control sample. After PBS washing, the cells were placed in DMEM and observed under a laser scanning confocal microscope. The changes in the fluorescence brightness of LysoSensor Green in cells from the two groups were recorded.

### Result Analysis:

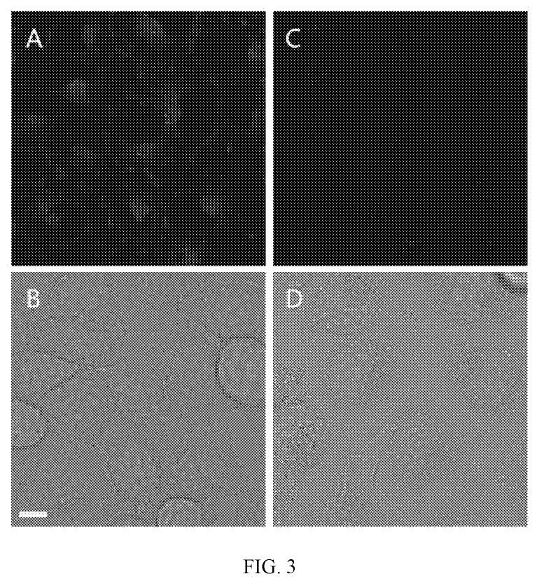

The experimental results of Test example 4 are shown in FIG. 3. FIG. 3 shows fluorescence micrographs of the lysosomal green fluorescent probe (LysoSensor Green DND-189) in HeLa cells before and after treatment with the compound synthesized in Example 1. FIG. 3(A) shows a fluorescence micrograph of LysoSensor Green in cells from the blank control sample group; FIG. 3(B) shows a bright-field micrograph corresponding to FIG. 3(A); FIG. 3(C) shows a fluorescence micrograph of LysoSensor Green in the cells treated with the compound synthesized in Example 1; FIG. 3(D) shows a bright-field micrograph corresponding to FIG. 3(C). The fluorescence of LysoSensor Green DND-189 increases as pH decreases. It can be learned from FIG. 3(A) and FIG. 3(C) that, in the cells treated with the compound synthesized in Example 1, the fluorescence of LysoSensor Green is greatly weakened (FIG. 3(C)). This represents that the lysosomal pH of the cells treated with the compound synthesized in Example 1 increases significantly, indicating lysosomal dysfunction in autophagic flux.

### Test Example 5

Imaging of Autophagic Vacuoles and Autophagosomes in HeLa Cells Before and After Treatment with (E)-4-(2-(5-Methoxy-1H-Indol-3-Yl)Vinyl)-1-n-Dodecylquinolinium Iodide

Two groups of HeLa cells grown on confocal glass-bottom culture dishes prepared in Test example 1 were firstly washed with PBS. Then one group was incubated in the dark for 90 min in complete medium with 10 μM of the compound synthesized in Example 1, and the other group was incubated in complete medium with an equal amount of DMSO as a blank control sample. Subsequently, the cells were incubated in DMEM with 5 μM green live-cell tracking probe (Cell-Tracker Green CMFDA) for 30 min. After PBS washing, the cells were placed in DMEM and observed under a laser scanning confocal microscope. The fluorescence distribution of Cell-Tracker Green in cells from the two groups was recorded.

### Result Analysis:

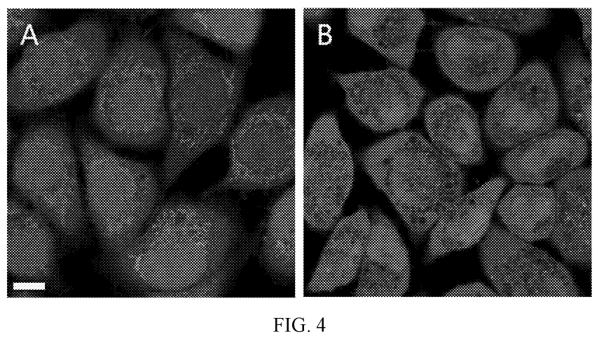

The experimental results of Test example 5 are shown in FIG. 4. FIG. 4 shows fluorescence micrographs of Cell-Tracker Green in HeLa cells before and after treatment with the compound synthesized in Example 1. FIG. 4(A) shows a fluorescence micrograph of Cell-Tracker Green in cells from the blank control sample group; FIG. 4(B) shows a fluorescence micrograph of Cell-Tracker Green in cells treated with the compound synthesized in Example 1. Comparison of FIG. 4(A) and FIG. 4(B) shows that a large number of vacuoles (autophagic vacuoles and autophagosomes) are generated in the cytoplasm of the cells treated with the compound synthesized in Example 1, indicating that the molecule synthesized in Example 1 can activate autophagy.

### Test Example 6

Fusion of Mitochondria and Lysosomes in HeLa Cells Treated with (E)-4-(2-(5-Methoxy-1H-Indol-3-Yl)Vinyl)-1-n-Dodecylquinolinium Iodide

After two groups of HeLa cells grown on confocal glass-bottom culture dishes prepared in Test example 1 were washed with PBS, both were incubated with 1 μM mitochondrial green fluorescent probe (MitoTracker Green) in the dark for 30 min, followed by 30 min incubation with 1 μM lysosomal deep-red probe (LysoBrite NIR). Subsequently, one group was incubated with 20 μM CCCP (mitophagy inducer) in PBS for ~5 h as a mitophagy positive control sample, and the other group was incubated in the dark for the same period in complete medium with 10 μM of the compound synthesized in Example 1. After PBS washing, the cells were placed in DMEM and observed under a laser scanning confocal microscope. The fluorescence distribution of MitoTracker Green and LysoBrite NIR in cells from the two groups was recorded.

### Result Analysis:

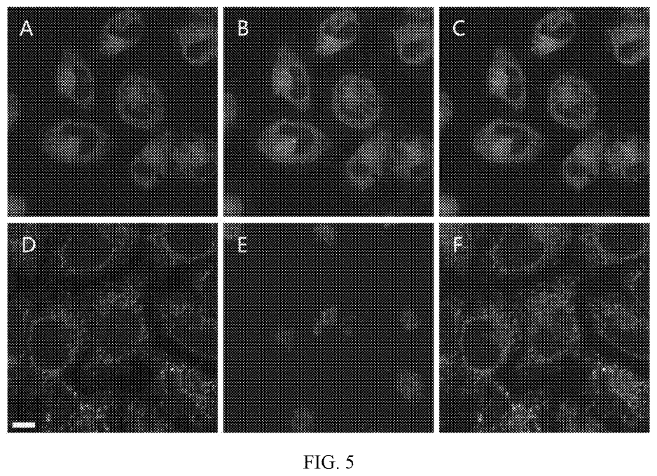

The experimental results of Test example 6 are shown in FIG. 5. FIG. 5 shows fluorescence micrographs of mitochondria and lysosomes in HeLa cells after treatment with the molecule synthesized in Example 1. FIG. 5(A) shows a mitochondrial fluorescence micrograph of cells in the positive control sample group; FIG. 5(B) shows a lysosomal fluorescence micrograph of cells in the positive control sample group; FIG. 5(C) shows an overlay image of FIG. 5(A) and FIG. 5(B); FIG. 5(D) shows a mitochondrial fluorescence micrograph of cells treated with the compound synthesized in Example 1; FIG. 5(E) shows a lysosomal fluorescence micrograph of cells treated with the compound synthesized in Example 1; FIG. 5(F) shows an overlay image of FIG. 5(D) and FIG. 5(E). The fluorescence of FIG. 5(A) and FIG. 5(B) overlaps very well, indicating that mitochondria and lysosomes fuse in the late stage of mitophagy; the fluorescence of FIG. 5(D) and FIG. 5(E) shows almost no overlap, indicating that after autophagy is activated by the compound synthesized in Example 1, mitochondria and lysosomes cannot fuse in the late stage of autophagy. Comparison analysis of FIG. 5(C) and FIG. 5(F) indicates that the compound synthesized in Example 1 can prevent fusion of cellular mitochondria and lysosomes and block autophagic flux.

### Test Example 7

### Verification of how (E)-4-(2-(5-Methoxy-1H-Indol-3-Yl)Vinyl)-1-n-Dodecylquinolinium Iodide Enters Cells

After two groups of HeLa cells grown on confocal glass-bottom culture dishes prepared in Test example 1 were washed with PBS, one group was incubated at 37 °C in the dark for 30 min in complete medium with 2 μM of the compound synthesized in Example 1, and the other group was incubated at 4 °C for the same period in complete medium with 2 μM of compound synthesized in Example 1. After PBS washing, the incubated cells were placed in DMEM, and red fluorescence distribution in cells from the two groups was observed and recorded using a laser scanning confocal microscope (EX561 nm, EM600-700 nm).

### Result Analysis:

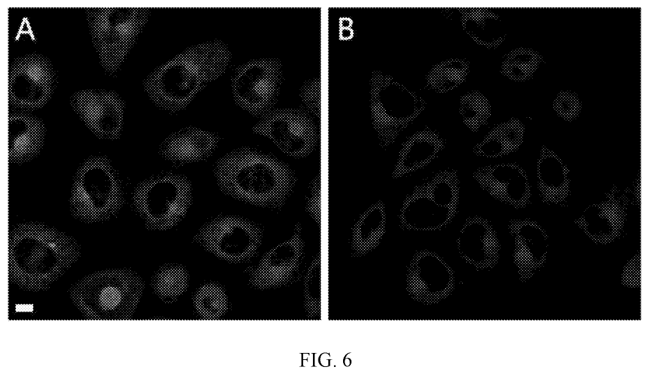

The experimental results of Test example 7 are shown in FIG. 6. FIG. 6(A) and FIG. 6(B) show red fluorescence micrographs of HeLa cells incubated with (E)-4-(2-(5-methoxy-1H-indol-3-yl)vinyl)-1-n-dodecylquinolinium iodide at 37 °C and 4 °C, respectively. Comparison shows that the fluorescence of FIG. 6(A) is significantly stronger than the fluorescence of FIG. 6(B), indicating that (E)-4-(2-(5-methoxy-1H-indol-3-yl)vinyl)-1-n-dodecylquinolinium iodide enters cells mainly via active transport.

### Test Example 8

Changes in the Growth of HEK293 and HeLa Cell Spheroids after Administration of (E)-4-(2-(5-Methoxy-1H-Indol-3-Yl)Vinyl)-1-n-Dodecylquinolinium Iodide

After two groups of HEK293 cell spheroids and two groups of HeLa cell spheroids prepared in Test example 1 were photographed under a microscope, one group of HEK293 and HeLa cell spheroids was incubated in a complete medium containing 10 μM of the compound synthesized in Example 1 as an experimental administration group, and the other group of HEK293 and HeLa cell spheroids was incubated in a complete medium containing the same volume of DMSO as a blank control. After 24 h, photographs were taken again using the microscope to observe changes in morphology and size of the cell spheroids.

### Result Analysis:

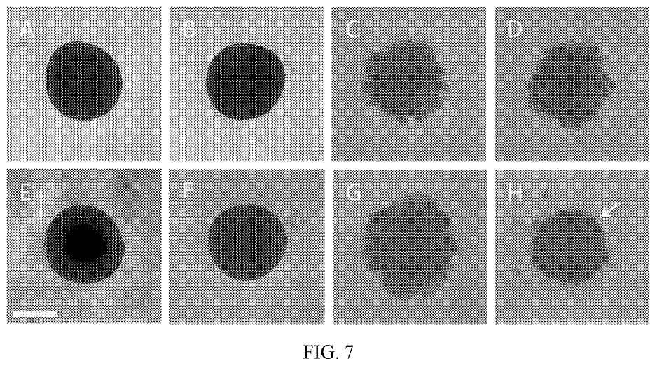

The experimental results of Test example 8 are shown in FIG. 7. FIG. 7(A) and FIG. 7(B) show bright-field micrographs of HEK293 cell spheroids in the blank control and the administration group before administration, respectively; FIG. 7(C) and FIG. 7(D) show bright-field micrographs of HeLa cell spheroids in the blank control and the administration group before administration, respectively; FIG. 7(E) and FIG. 7(G) show bright-field micrographs of HEK293 cell spheroids and HeLa cell spheroids, respectively, in the blank control after 24 h incubation; FIG. 7(F) and FIG. 7(H) show bright-field micrographs of HEK293 cell spheroids and HeLa cell spheroids, respectively, in the administration group after administration for 24 h. Comparison of FIG. 7(A), FIG. 7(B), FIG. 7(E) and FIG. 7(F) shows that the size and shape of HEK293 cell spheroids in the experimental administration group do not change significantly compared with the blank control, indicating that 10 μM of the compound synthesized in Example 1 does not cause significant growth inhibition on normal HEK293 cell spheroids. Whereas, comparison of FIG. 7(C), FIG. 7(D), FIG. 7(G) and FIG. 7(H) shows that HeLa cell spheroids in the blank control become significantly larger after 24 h while HeLa cell spheroids in the administration group after 24 h are much smaller than those in the blank control as well as than the cell spheroids before drug administration, and many cell debris appear near the cell spheroids, indicating that 10 μM of the compound synthesized in Example 1 significantly inhibits and disrupts the growth of cancerous HeLa cell spheroids. The results show that the compound synthesized in Example 1 exhibits a significant selective killing effect on cancer cells.

### Test Example 9

### Binding Constant Between (E)-4-(2-(5-Methoxy-1H-Indol-3-Yl)Vinyl)-1-n-Dodecylquinolinium Iodide and Albumin

The compound synthesized in Example 1 was added at different concentrations (0 to 15 μM) to PBS containing bovine serum albumin, and then fluorescence spectra of bovine serum albumin were measured using a fluorescence spectrometer (280 nm EX).

### Result Analysis:

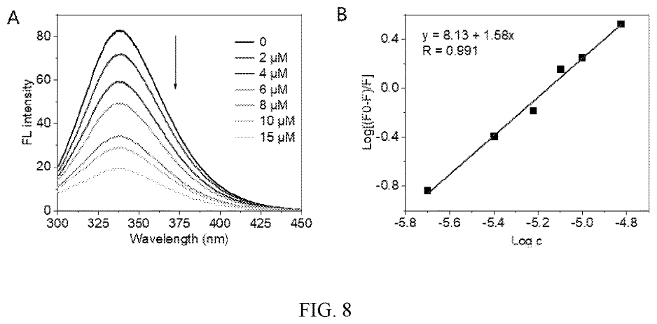

The experimental results of Test example 9 are shown in FIG. 8. FIG. 8(A) shows fluorescence spectra of bovine serum albumin with the addition of the compound synthesized in Example 1 at different concentrations (0 to 15 μM), and FIG. 8(B) shows a double-logarithmic fitting curve of the degree of fluorescence intensity decrease versus the concentration of the compound synthesized in Example 1 added, made according to FIG. 8(A). It can be seen from FIG. 8(A) that, as the concentration of the compound synthesized in Example 1 added increases, the fluorescence peak of bovine serum albumin gradually decreases. According to the fitting curve in FIG. 8(B), the binding constant between the compound synthesized in Example 1 and albumin is as high as 1.35×10⁸, significantly greater than 10⁶, indicating that the compound synthesized in Example 1 has high affinity for albumin.

### Test Example 10

### Selectivity of (E)-4-(2-(5-Methoxy-1H-Indol-3-Yl)Vinyl)-1-n-Dodecylquinolinium Iodide and (E)-4-(2-(5-Methoxy-1H-Indol-3-Yl)Vinyl)-1-n-Octylquinolinium Iodide for Tumor Cells in Complete Medium

After three groups of cancer cells HeLa and 4T1 and normal cells HEK293 individually grown on confocal glass-bottom culture dishes prepared in Test example 1 were washed with PBS, one group was incubated in complete medium (serum-containing) with 1 μM of the compound synthesized in Example 1 in the dark for 30 min, and the second group was incubated in complete medium with 1 μM of (E)-4-(2-(5-methoxy-1H-indol-3-yl)vinyl)-1-n-octylquinolinium iodide in the dark for 30 min, and the third group was incubated in DMEM (serum-free)with 1 μM of the compound synthesized in Example 1 in the dark for 30 min. After PBS washing, all the cells were placed in DMEM, and differences in red fluorescence intensity from the three groups of tumor cells and normal cells were observed and recorded under a laser scanning confocal microscope (EX561 nm, EM600-700 nm).

### Result Analysis:

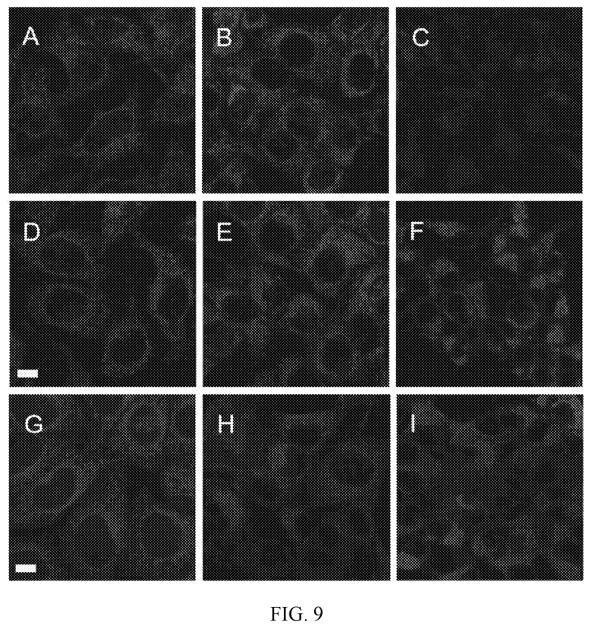

The experimental results of Test example 10 are shown in FIG. 9. FIG. 9 shows fluorescence micrographs of cancer cells HeLa and 4T1 and normal cells HEK293 after incubation with the compound synthesized in Example 1 and (E)-4-(2-(5-methoxy-1H-indol-3-yl)vinyl)-1-n-octylquinolinium iodide in complete medium and DMEM (serum-free). FIG. 9(A) shows a fluorescence micrograph of HeLa cells after incubation with the compound synthesized in Example 1 in complete medium; FIG. 9(B) shows a fluorescence micrograph of 4T1 cells after incubation with the compound synthesized in Example 1 in complete medium; FIG. 9(C) shows a fluorescence micrograph of HEK293 cells after incubation with the compound synthesized in Example 1 in complete medium; FIG. 9(D) shows a fluorescence micrograph of HeLa cells after incubation with (E)-4-(2-(5-methoxy-1H-indol-3-yl)vinyl)-1-n-octylquinolinium iodide in complete medium; FIG. 9(E) shows a fluorescence micrograph of 4T1 cells after incubation with (E)-4-(2-(5-methoxy-1H-indol-3-yl)vinyl)-1-n-octylquinolinium iodide in complete medium; FIG. 9(F) shows a fluorescence micrograph of HEK293 cells after incubation with (E)-4-(2-(5-methoxy-1H-indol-3-yl)vinyl)-1-n-octylquinolinium iodide in complete medium. FIG. 9(A-C) show that after incubation with the compound synthesized in Example 1 in complete medium, the fluorescence intensity of cancer cells is significantly higher than the fluorescence intensity of normal cells; FIG. 9(D-F) show that there is no significant difference in fluorescence intensity between cancer cells and normal cells incubated with (E)-4-(2-(5-methoxy-1H-indol-3-yl)vinyl)-1-n-octylquinolinium iodide in complete medium. These indicate that the compound synthesized in Example 1 exhibits high selectivity for cancer cells, whereas (E)-4-(2-(5-methoxy-1H-indol-3-yl)vinyl)-1-n-octylquinolinium iodide is non-selective for cancer cells.

FIG. 9(G) shows a fluorescence micrograph of HeLa cells after incubation with the compound synthesized in Example 1 in DMEM; FIG. 9(H) shows a fluorescence micrograph of 4T1 cells after incubation with the compound synthesized in Example 1 in DMEM; FIG. 9(I) shows a fluorescence micrograph of HEK293 cells after incubation with the compound synthesized in Example 1 in DMEM. FIG. 9(G-I) show that after incubation with the compound synthesized in Example 1 in DMEM (serum-free), there is almost no difference in fluorescence intensity between cancer cells and normal cells. This indicates that the compound synthesized in Example 1 enters cancer cells and normal cells in a serum-free medium, showing no significant difference.

### Test Example 11

### Verification of how (E)-4-(2-(5-Methoxy-1H-Indol-3-Yl)Vinyl)-1-n-Octylquinolinium Iodide Enters Cells

After two groups of HeLa cells grown on confocal glass-bottom culture dishes prepared in Test example 1 were washed with PBS, one group was incubated in complete medium with 2 μM of (E)-4-(2-(5-methoxy-1H-indol-3-yl)vinyl)-1-n-octylquinolinium iodide in the dark at 37 °C for 30 min, and the other group was incubated in complete medium solution with 2 μM (E)-4-(2-(5-methoxy-1H-indol-3-yl)vinyl)-1-n-octylquinolinium iodide in the dark at 4 °C for the same period. After PBS washing, the cells were placed in DMEM, and red fluorescence in cells from the two groups was observed and recorded using a laser scanning confocal microscope (EX561 nm, EM600-700 nm).

### Result Analysis:

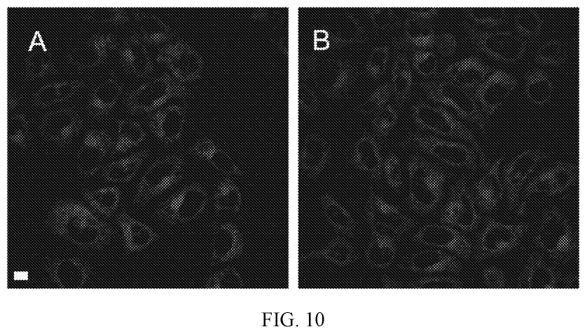

The experimental results of Test example 11 are shown in FIG. 10. FIG. 10(A) and FIG. 10(B) show red fluorescence micrographs of HeLa cells incubated with (E)-4-(2-(5-methoxy-1H-indol-3-yl)vinyl)-1-n-octylquinolinium iodide at 37 °C and 4 °C, respectively. Comparison shows that there is no obvious difference in fluorescence intensity between FIG. 10(A) and FIG. 10(B). This indicates that (E)-4-(2-(5-methoxy-1H-indol-3-yl)vinyl)-1-n-octylquinolinium iodide enters cells mainly via free diffusion rather than active transport, and it enters cells differently from the compound synthesized in Example 1 of the present disclosure.

### Test Example 12

### Localization Observation of (E)-4-(2-(1H-Indol-3-Yl)Vinyl)-1-n-Dodecylquinolinium Iodide in HeLa Cells

After HeLa cells grown on confocal glass-bottom culture dish prepared in Test example 1 were washed twice with PBS, the following staining steps were carried out: (1) incubation with 0.5 μM commercial mitochondrial green fluorescent probe (MitoTracker Green) for 30 min, followed by PBS washing; (2) incubation with 0.5 μM commercial lysosomal near-infrared fluorescent probe (LysoBrite NIR) for 30 min, followed by PBS washing; (3) incubation with 2 μM (E)-4-(2-(1H-indol-3-yl)vinyl)-1-n-dodecylquinolinium iodide fluorescent probe for 30 min, followed by DMEM washing. The stained cells were subjected to multichannel fluorescence observation using a confocal fluorescence microscope (channel 1: EX488 nm/EM490-530 nm, channel 2: EX638 nm/EM660-750 nm, channel 3: EX561 nm/EM580-650 nm).

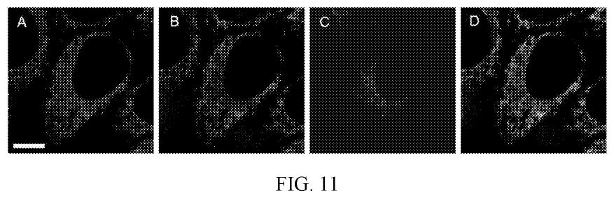

The results are shown in FIG. 11. FIG. 11(A) shows a red fluorescence image from the compound synthesized in Example 2; FIG. 11(B) shows a fluorescence image from commercial MitoTracker Green; FIG. 11(C) shows a fluorescence image from commercial LysoBrite NIR; FIG. 11(D) shows an overlay image of FIG. 11(A), FIG. 11(B) and FIG. 11(C). FIG. 11(A) covers both regions of FIG. 11(B) and FIG. 11(C) and overlaps excellent with the combined signal of FIG. 11(B) and FIG. 11(C), indicating that the fluorescence of the compound synthesized in Example 2 is distributed in the two organelles, mitochondria and lysosomes. This result demonstrates that (E)-4-(2-(1H-indol-3-yl)vinyl)-1-n-dodecylquinolinium iodide can be used for targeting mitochondria and lysosomes as well as simultaneous fluorescence imaging of mitochondria and lysosomes.

### Test Example 13

Imaging of Autophagic Vacuoles and Autophagosomes in HeLa Cells Before and After Treatment with (E)-4-(2-(1H-Indol-3-Yl)Vinyl)-1-n-Dodecylquinolinium Iodide

After HeLa cells grown on confocal glass-bottom culture dish prepared in Test example 1 was washed with PBS, they were incubated in complete medium with 10 μM of the compound synthesized in Example 2 in the dark for 90 min, and then incubated in DMEM with 5 μM green live-cell tracking probe (Cell-Tracker Green CMFDA) in the dark for 30 min followed by PBS washing. The cells were placed in DMEM and observed under a laser scanning confocal microscope. The fluorescence distribution of Cell-Tracker Green in the cells was recorded.

### Result Analysis:

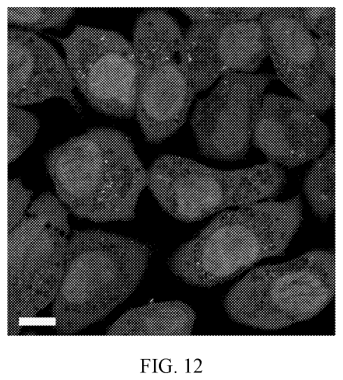

The experimental results of Test example 13 are shown in FIG. 12. FIG. 12 shows a fluorescence micrograph of Cell-Tracker Green in HeLa cells after treatment with the compound synthesized in Example 2. FIG. 12 shows that a large number of vacuoles (autophagic vesicles and autophagosomes) are generated in the cytoplasm in the cells treated with the compound synthesized in Example 2, demonstrating the autophagy-modulating activity of the compound synthesized in Example 2.

In summary, the present application provides a dual-targeting fluorescent organic small-molecule autophagy regulator and use thereof. Compared with existing autophagy activators and autophagy inhibitors, the dual-targeting fluorescent organic small-molecule autophagy regulator of the present disclosure can simultaneously target mitochondria and lysosomes, on the one hand damaging mitochondria to activate mitophagy, and on the other hand alkalinizing lysosomal pH and preventing fusion of mitochondria and lysosomes to block autophagic flux. With no light assistance, the effect of killing cancer cells can be achieved with autophagy regulator of the present disclosure by activating autophagy and blocking autophagic flux, and cancer cells can be selectively killed in the presence of serum or albumin. In addition, the fluorescence of autophagy regulator of the present disclosure enhances greatly after targeting mitochondria and lysosomes, thereby realizing synchronous fluorescence visualization of mitochondria and lysosomes.

The above descriptions are only preferred examples of the present application and are not intended to limit the scope thereof. Any modifications, equivalent substitutions, improvements, etc. made within the spirit and principles of the present application shall be included within the protection scope of the present application.

What is claimed is:

1. An autophagy regulator, wherein the autophagy regulator is a compound having a structure represented by formula (I): 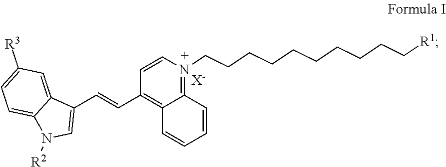
wherein the R¹ is selected from hydrogen, methyl, ethyl, and n-propyl; the R² is selected from hydrogen, methyl, ethyl, and n-propyl; the R³ is selected from hydrogen, methyl, ethyl, n-propyl, methoxy, ethoxy, and n-propoxy; and the X is selected from halogen atom, BF₄, and ClO₄.

2. The autophagy regulator according to claim 1, wherein the autophagy regulator simultaneously targets mitochondria and lysosomes, activates mitophagy and blocks autophagic flux by disrupting lysosomal function.

3. The autophagy regulator according to claim 2, wherein after the autophagy regulator targets mitochondria, the mitochondrial membrane potential decreases.

4. The autophagy regulator according to claim 2, wherein after the autophagy regulator targets lysosomes, the pH value of the lysosomes increases.

5. The autophagy regulator according to claim 1, wherein the autophagy regulator targets mitochondria and/or lysosomes of a sample cell, the fluorescence enhances and fluorescence imaging of the mitochondria and/or lysosomes is achieved.

6. The autophagy regulator according to claim 1, wherein the autophagy regulator comprises (E)-4-(2-(5-methoxy-1H-indol-3-yl)vinyl)-1-n-dodecylquinolinium iodide or (E)-4-(2-(1H-indol-3-yl)vinyl)-1-n-dodecylquinolinium iodide.

7. The autophagy regulator according to claim 6, wherein the autophagy regulator exhibits high affinity for albumin, and after binding to albumin, can selectively enter a cancer cell via active transport and selectively kill the cancer cell.

8. A product for cellular autophagy regulation, wherein the product comprises the autophagy regulator according to claim 1.

9. The product for cellular autophagy regulation according to claim 8, wherein the autophagy regulator is (E)-4-(2-(5-methoxy-1H-indol-3-yl)vinyl)-1-n-dodecylquinolinium iodide or (E)-4-(2-(1H-indol-3-yl)vinyl)-1-n-dodecylquinolinium iodide.

10. A product for targeting mitochondria and/or targeting lysosomes, wherein the product comprises the autophagy regulator according to claim 1.

11. The product for targeting mitochondria and/or targeting lysosomes according to claim 10, wherein the autophagy regulator is (E)-4-(2-(5-methoxy-1H-indol-3-yl)vinyl)-1-n-dodecylquinolinium iodide or (E)-4-(2-(1H-indol-3-yl)vinyl)-1-n-dodecylquinolinium iodide.

12. A product for mitochondrial imaging and/or lysosomal imaging, wherein the product comprises the autophagy regulator according to claim 1.

13. The product for mitochondrial imaging and/or lysosomal imaging according to claim 12, wherein the autophagy regulator is (E)-4-(2-(5-methoxy-1H-indol-3-yl)vinyl)-1-n-dodecylquinolinium iodide or (E)-4-(2-(1H-indol-3-yl)vinyl)-1-n-dodecylquinolinium iodide.

14. A drug for tumor treatment, wherein the drug comprises the autophagy regulator according to claim 1.

15. The drug for tumor treatment according to claim 14, wherein the autophagy regulator is (E)-4-(2-(5-methoxy-1H-indol-3-yl)vinyl)-1-n-dodecylquinolinium iodide or (E)-4-(2-(1H-indol-3-yl)vinyl)-1-n-dodecylquinolinium iodide.
